(19) United States
(12) Patent Application Publication
(10) Pub. No.: US 2017/0086889 A1
(43) Pub. Date: Mar. 30, 2017

(54) SPINAL IMPLANT SYSTEM AND METHOD

(71) Applicant: Warsaw Orthopedic, Inc., Warsaw, IN (US)

(72) Inventors: Maria-Rosa Padilla, Nesbit, MS (US); Joshua W. Simpson, Collierville, TN (US)

(21) Appl. No.: 14/864,507

(22) Filed: Sep. 24, 2015

(51) Int. Cl. A61B 17/70

(52) U.S. Cl. CPC A61B 17/7053; A61B 2017/00946

(57) ABSTRACT

A spinal implant comprises a body including a support and a base that define a cavity configured for disposal of a member and including a wall that defines a bay configured for disposal of a longitudinal dement. The longitudinal element is engageable with the wall to fix the longitudinal dement with the body and move the base relative to the support to fix the member with the body in the cavity. Systems and methods are disclosed.

### TECHNICAL HELD

The present disclosure generally relates to medical devices for the treatment of musculoskeletal disorders, and more particularly to a surgical system and method for correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, bone screws and sub-laminar wire, for stabilization of a treated section of a spine. This disclosure describes an improvement over these prior technologies.

### SUMMARY

In one embodiment, a spinal implant comprises a body including a support and a base that define a cavity configured for disposal of a member and including a wall that defines a bay configured for disposal of a longitudinal element. The longitudinal element is engageable with the wall to fix the longitudinal element with the body and move the base relative to the support to fix the member with the body in the cavity. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of one embodiment of components of a surgical system in accordance with the principles of the present disclosure;

FIG. 2 is a side view of the components shown in FIG. 1;

FIG. 3 is a side view of the components shown in FIG. 1;

FIG. 4 is a perspective view of one embodiment of components of a surgical system in accordance with the principles of the present disclosure;

FIG. 5 is a perspective view of the components shown in FIG, 1;

FIG. 6 is a side view of one embodiment of components of a surgical system in accordance with the principles of the present disclosure;

FIG. 7 is a perspective view of one embodiment of components of a surgical system in accordance with the principles of the present disclosure;

FIG. 8 is a perspective view of one embodiment of components of a surgical system in accordance with the principles of the present disclosure; and

FIG. 9 is a perspective view of one embodiment of components of a surgical system in accordance with the principles of the present disclosure disposed with vertebrae.

### DETAILED DESCRIPTION

The exemplary embodiments of a surgical system and related methods of use are discussed in terms of medical devices for the treatment of musculoskeletal disorders and more particularly, in terms of a surgical system and method for correction of a spine disorder. In some embodiments, the surgical system may be employed in applications for correction of deformities, such as scoliosis and kyphosis.

In some embodiments, the surgical system includes a spinal implant including a sub-laminar tether connector. In some embodiments, the surgical system includes a spinal implant including a tether connector configured to fix a posterior spinal rod to a spine by a tether such that the spinal rod is fixed in a flexible and/or dynamic configuration. In some embodiments, the tether connector is fixed with a spine at a top level of a spinal construct. In some embodiments, the tether connector is fixed with a spine at one or a plurality of levels of a spinal construct. In some embodiments, the tether connector is fixed to a spinal rod and maintains a tether in place after the tether is wrapped about vertebral tissue, such as, for example, a lamina.

In some embodiments, the tether connector includes one or a plurality of coupling members, such as, for example, set screws. In some embodiments, the tether connector includes a set screw that fixes a spinal rod in position with the tether connector such that the tether connector also fixes the tether in position. In some embodiments, the tether connector includes a single set screw that fixes a spinal rod in position with the tether connector such that the tether connector also fixes the tether in position. In some embodiments, the tether connector includes an adjustable rod slot that accommodates spinal rods of various diameters. In some embodiments, the tether connector includes a provisional retention element, such as, for example, a snap on element. In some embodiments, the tether connector includes a provisional retention element that temporarily holds a spinal rod in place while a tether is being tightened. In some embodiments, the provisional retention element includes a finger.

In some embodiments, the spinal implant includes a tether connector having a member, such as, for example, a base connected with a member, such as, for example, a support. In some embodiments, the spinal implant includes a hinged sub-laminar tether connector, which utilizes a single set screw to secure a tether to a spinal rod. In some embodiments, tightening the set screw causes the spinal rod to engage on a bottom section of the tether connector, which causes the members of the tether connector to splay apart. In some embodiments, the splaying of the members forces the tether connector to compress against the tether. The set screw can then be tightened such that the tether is secured to the spinal rod.

In some embodiments, the surgical system is employed with a method including the step of opening the tether connector via a hinge. In some embodiments, the method includes the step of passing the tether through a tether slot of the tether connector. In some embodiments, the method includes the step of tightening a set screw of the tether connector such that a spinal rod disposed with a rod slot of the tether connector engages a bottom section of the tether connector such that members of the tether connector splays apart. In some embodiments, the method includes the step of relatively moving the members of the tether connector to force the members to compress against the tether such that the tether is secured to the spinal rod via the connector.

In some embodiments, the tether connector includes a fixation surface, such as, for example, a toothed and/or roughened surface adjacent a tether slot of the tether connector. In some embodiments, the fixation surface engages a tether and resists and/or prevents tether slippage when the tether connector is tightened. In some embodiments, the tether connector includes members such that tether flexibility creates a provisional retention element, such as, for example, a snap on element when pushing a spinal rod into a rod slot of the tether connector. In some embodiments, this configuration of the tether connector provides engagement with the spinal rod without a set screw and allows translation and/or sliding of the tether connector along and/or about the spinal rod.

In some embodiments, the surgical system is used with surgical navigation, such as, for example, fluoroscope or image guidance. In one embodiment, one or all of the components of the surgical system are disposable, peel-pack, pre-packed sterile devices. One or all of the components of the surgical system may be reusable. The surgical system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e,g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. As used in the specification and including the appended claims, the term “tissue” includes soft tissue, ligaments, tendons, cartilage and/or bone unless specifically referred to otherwise,

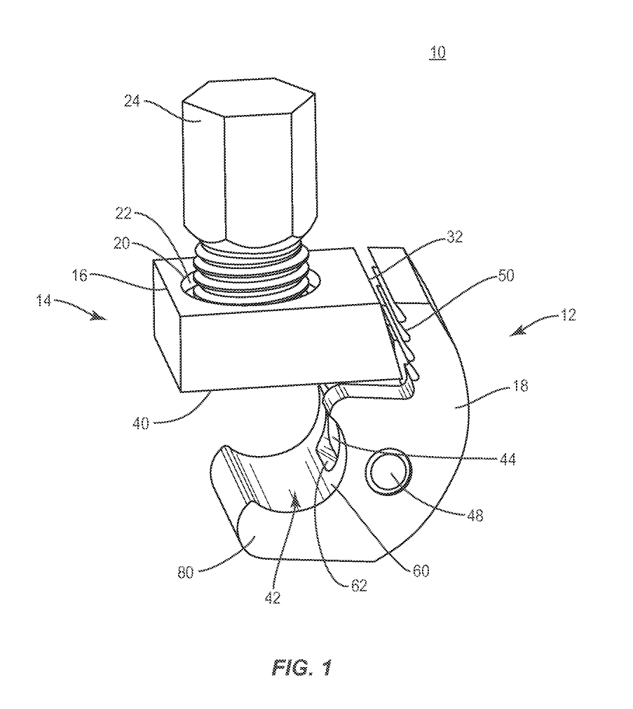

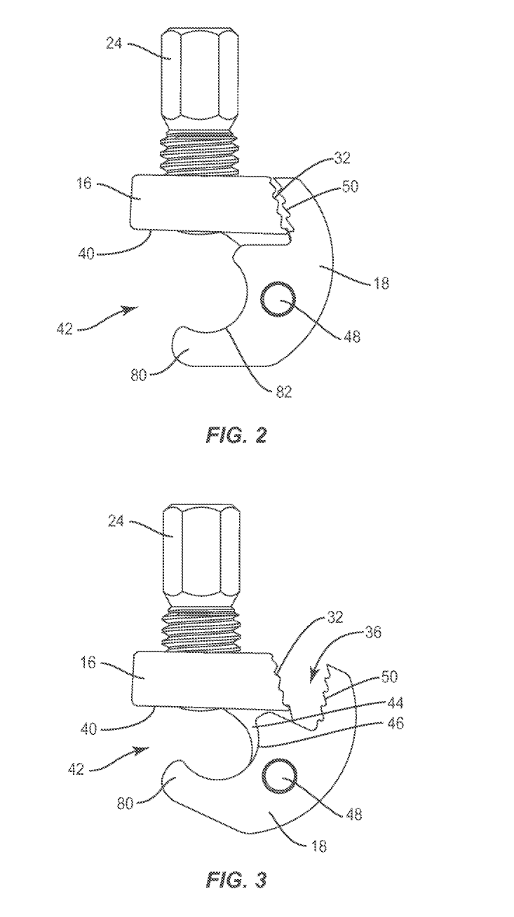

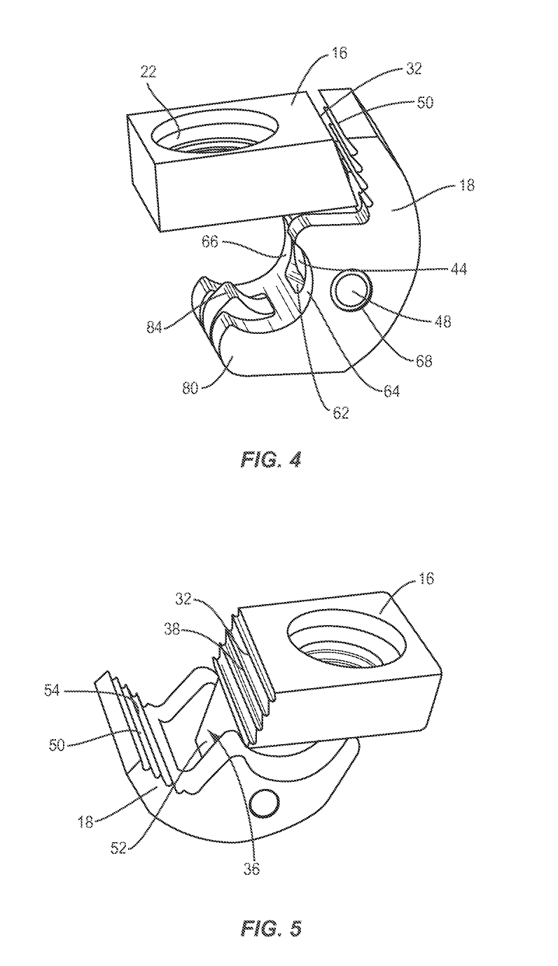

The following discussion includes a description of a surgical system and related methods of employing the surgical system in accordance with the principles of the present disclosure. Alternate embodiments are also disclosed. Reference is made in detail to exemplary embodiments of the present disclosure, which are illustrated in the accompanying figures. Turning to FIGS. 1-6, there are illustrated components of a surgical system, such as, for example, a spinal implant system 10.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

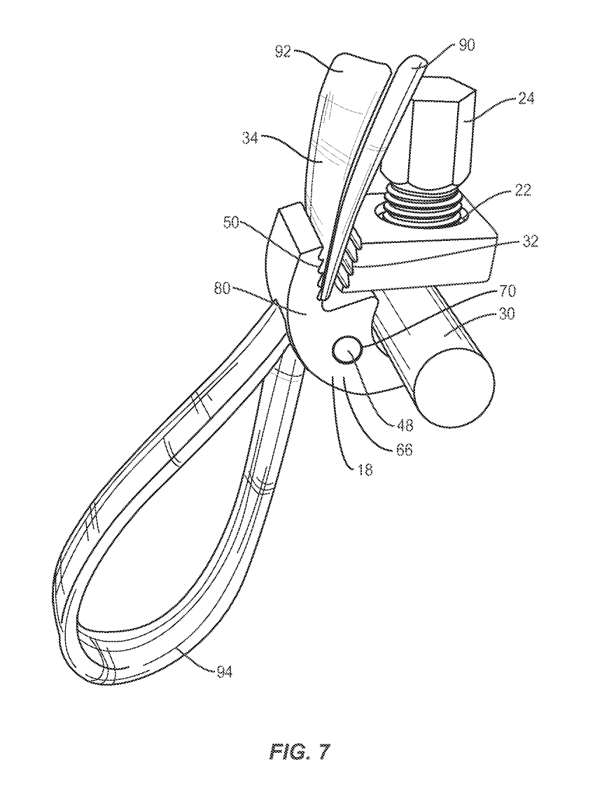

Spinal implant system 10 comprises a spinal implant, such as, for, example, a connector 12. Connector 12 includes a body 14 having a support 16 and a base 18. Support 16 includes a surface 20 that defines a cavity, such as, for example, an opening 22. Surface 20 is threaded and configured for disposal of a coupling member, such as, for example, a set screw 24. Set screw 24 is configured for engagement with a surgical instrument, such as, for example, a surgical driver (not shown). Set screw 24 is configured for engagement with a longitudinal element, such as, for example, a spinal rod 30 (FIG. 7).

In some embodiments, set screw 24 includes an end having a hexagonal geometry configured for engagement with a similarly shaped tool, such as, for example, a surgical driver. In some embodiments, set screw 24 includes an end having a cruciform, phillips, square, hexalobe, polygonal or star cross sectional configuration for disposal of a correspondingly shaped portion of a surgical driver. In some embodiments, set screw 24 is fabricated from a fracturing and/or frangible material such that manipulation of a portion of set screw 24 can fracture and separate the portion at a predetermined force and/or torque limit.

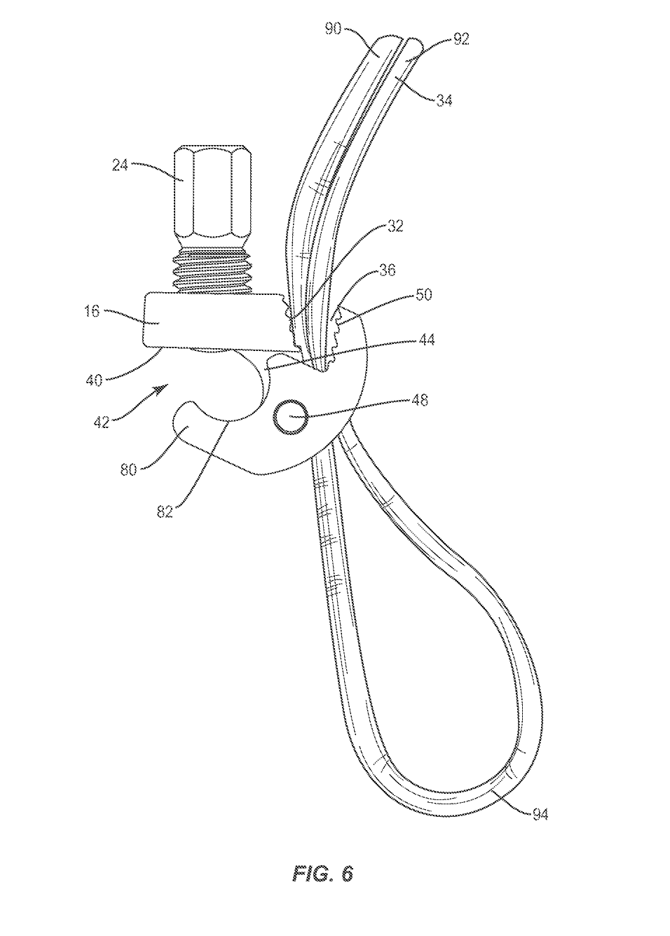

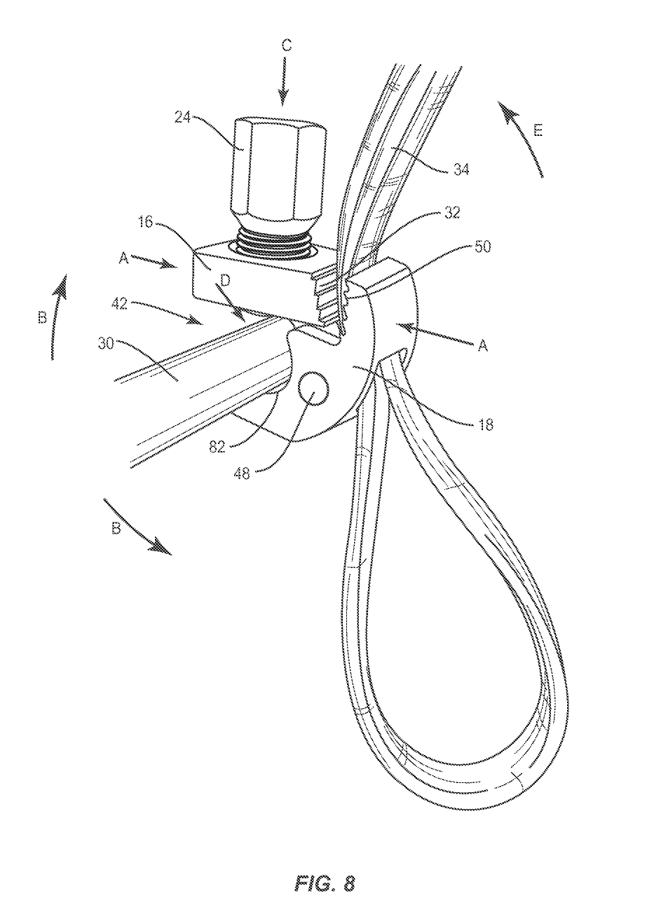

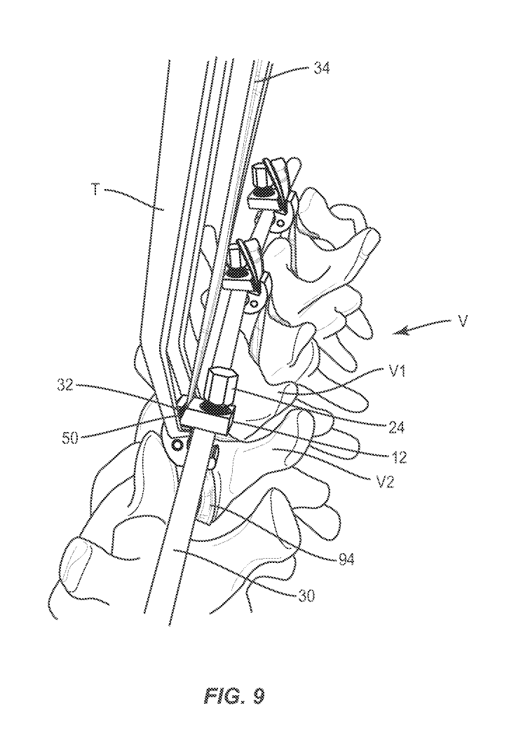

Support 16 includes a surface 32. Surface 32 defines an engagement surface and/or a fixation surface configured to facilitate fixation of a member, such as, for example, a tether 34 (FIG. 7) with body 14, as described herein. Surface 32 defines a portion of a cavity 36 configured for disposal of tether 34, as described herein. Tether 34 is configured for fixation with surface 32 and a surface of base 18, as described herein, via compressive forces and/or friction forces, applied at least in the direction shown by arrows A in FIG. 8. Tether 34 is configured for disposal between a non-locking orientation, such as, for example, as shown in FIGS. 6 and 7 and a locked orientation, such as, for example, as shown in FIG. 8.

In some embodiments, the compressive forces and/or friction forces applied by surface 32 may be directed in various orientations, such as, for example, perpendicular, transverse and/or at angular orientations, such as acute or obtuse. In some embodiments, surface 32 may include penetrating members, such as, for example, a plurality of teeth 38. In some embodiments, teeth 38 may have various configurations, for example, round, oval, rectangular, polygonal, irregular, tapered, offset, staggered, uniform and non-uniform. In some embodiments, surface 32 may include gripping elements or surfaces, such as, for example, rough, arcuate, undulating, mesh, porous, semi-porous, dimpled and/or textured to facilitate engagement with tether 34.

Support 16 includes a surface 40 that defines a portion of a cavity, such as, for example, a bay 42, as described herein. Bay 42 is configured for movable disposal of spinal rod 30, as described herein. Support 16 includes a protrusion, such as, for example, an arm 44 extending from surface 40. In some embodiments arm 44 may extend from surface 40 in various orientations, such as, for example, perpendicular, transverse and/or at angular orientations, such as acute or obtuse. Arm 44 is configured for connection with base 18 such that base 18 is rotatable relative to support 16. Arm 44 includes a surface 46 that defines an opening (now shown). The opening is configured for disposal of a pin hinge 48. Base 18 is configured to rotate about pin hinge 48 relative to support 16 to facilitate movement between a non-locking orientation and a locked orientation of one or more components of spinal implant system 10. In some embodiments, surface 40 may include gripping dements or surfaces, such as, for example, rough, arcuate, undulating, mesh, porous, semi-porous, dimpled and/or textured to facilitate engagement with spinal rod 30.

Base 18 includes a surface 50 that defines an engagement surface and/or a fixation surface configured to facilitate fixation of tether 34 with body 14, as described herein. Surface 50 defines cavity 36 with surface 32, as described herein. Surface 50 defines an opening 52 in communication with cavity 36, as shown in FIG. 5. Tether 34 is configured for disposal through opening 52 and into cavity 36 for fixation with surfaces 32, 50 via compressive forces and/or friction forces, as described herein.

In some embodiments, the compressive forces and/or friction forces applied by surface 50 may be directed in various orientations, such as, for example, perpendicular, transverse and/or at angular orientations, such as acute or obtuse. In some embodiments, surface 50 may include penetrating members, such as, for example, a plurality of teeth 54. In some embodiments, teeth 54 may have various configurations, for example, round, oval, rectangular, polygonal, irregular, tapered, offset, staggered, uniform and non-uniform. In some embodiments, surface 50 may include gripping elements or surfaces, such as, for example, rough, arcuate, undulating, mesh, porous, semi-porous, dimpled and/or textured to facilitate engagement with spinal rod 30.

Base 18 includes a surface 60 that defines a cavity 62. Cavity 62 is configured for movable disposal of arm 44. Cavity 62 includes a wall 64 and a wall 66. Wall 64 includes an opening 68 configured for disposal of pivot hinge 48. Wall 66 includes an opening 70 configured for disposal of pivot hinge 48. Arm 44 is configured to pivot within cavity 62 between a non-locking orientation and a locked orientation of one or more components of spinal implant system 10, as described herein.

Base 18 includes a wall 80 having a surface 82. Surface 82 and surface 40 define bay 42, which is configured for disposal of spinal rod 30. Spinal rod 30 is configured for adjustment of its spatial orientation, which can include translation, rotation, angular and/or pivot, relative to body 14. Spinal rod 30 is engageable with surface 82 and/or surface 40 between a non-locking orientation and a locked orientation of one or more components of spinal implant system 10, as described herein.

For example, tether 34 is disposed with body 14, as shown in FIG. 6, such that tether 34 is slidably movable within cavity 36. Spinal rod 30 is delivered and inserted into bay 42, as shown in FIG. 7. Spinal rod 30 is engageable with surface 82 and/or surface 40 to splay surfaces 82, 40 about hinge 48 to expand and/or open bay 42, in a direction shown by arrows B in FIG. 8. In some embodiments, this configuration may cause teeth 38, 54 (FIG. 5) to engage tether 34 and provisionally fix tether 34 with body 14.

Set screw 24 is engaged with support 16 via opening 22 in alignment with spinal rod 30. Set screw 24 is advanced, in the direction shown by arrow C in FIG. 8, for engaging spinal rod 30 such that spinal rod 30 translates within bay 42 to engage surface 82 with a force, in the direction shown by arrow D in FIG. 8. Spinal rod 30 engages surface 82 to rotate base 18 about hinge 48, in a direction shown by arrow E in FIG. 8. As set screw 24 is advanced and base 18 rotates, teeth 38, 54 engage tether 34 for fixation with surfaces 32, 50 via compressive forces and/or friction forces to dispose one or more components of spinal implant system 10 in a locked orientation. In some embodiments, set screw 24 is advanced and base 18 is selectively rotated such that teeth 38, 54 engage tether 34 for provisional fixation with surfaces 32, 50 to allow adjustment of one or more components of spinal implant system 10. In some embodiments, fixation of tether 34 with body 14 via rotation of base 18 and engagement of teeth 38, 54 with tether 34 fixes spinal rod 30, connector 12 and tether 34 in a selectively locked orientation in connection with a surgical treatment, as described herein. In some embodiments, base 18 and support 16 provisionally fix spinal rod 30 with body 14 in a selected orientation and/or such components may be more permanently fixed in a selected orientation thereafter.

In some embodiments, cavity 36 configured for disposal of tether 34 and is disposed separate from bay 42. In some embodiments, cavity 36 is disposed transverse relative to bay 42. In some embodiments, cavity 36 is disposed in various orientations relative to bay 42, such as, for example, perpendicular and/or at angular orientations, such as acute or obtuse.

In some embodiments, wall 80 includes a resilient configuration configured to snap fit with spinal rod 30. In some embodiments, surface 82 includes a protrusion, such as, for example, a finger 84, as shown in FIG. 4. In some embodiments, finger 84 includes a resilient configuration configured to snap fit with spinal rod 30. In some embodiments, finger 84 is configured to provisionally fix spinal rod 30 with body 14 to provisionally fix one or more components of spinal implant system 10 in a selected orientation and/or such components may be more permanently fixed in a selected orientation thereafter. In some embodiments, surface 82 can have cross-hatch texturing, spikes, barbs, raised elements, a porous titanium coating, and/or be rough, textured, porous, semi-porous, dimpled and/or polished.

In some embodiments, body 14 includes a mating surface (not shown) that defines cavities, such as, for example, mating capture elements (not shown) configured to mate with a surgical instrument to facilitate implant and manipulation of connector 12 and/or components of spinal implant system 10. In some embodiments, spinal implant system 10 may include one or a plurality of connectors 12 spaced apart and disposed along spinal rod 30, which may be relatively disposed in a side by side, irregular, uniform, non-uniform, offset and/or staggered orientation or arrangement, along one or a plurality of spinal rods 30. In some embodiments, spinal rod 30 extends along one or a plurality of vertebra, as described herein. In some embodiments, spinal implant system 10 may include one or a plurality of spinal rods 30, which may be relatively disposed in a side by side, irregular, uniform, non-uniform, offset and/or staggered orientation or arrangement.

Tether 34 extends between an end 90 and an end 92, as shown in FIG. 7. Tether 34 is configured for engagement with connector 12, as described herein. In some embodiments, end 90 and end 92 form a loop 94 configured to surround all or a portion of tissue, such as, for example, lamina tissue and/or transverse process, as described herein. Tether 34 is configured for tensioning about a targeted portion of an anatomy of a body for attachment of tether 34 with the targeted portion of the anatomy, as described herein. In some embodiments, the targeted portion of the anatomy may include laminae, transverse processes and/or pedicle regions of a vertebral level. In some embodiments, spinal implant system 10 may include one or a plurality of tethers 34, each tether 34 being configured for disposal about a single and separate vertebral level. In some embodiments, a single vertebral level may include one or a plurality of tethers 34.

Tether 34 has a flexible configuration and may be fabricated from materials, such as, for example, fabric, silicone, polyurethane, silicone-polyurethane copolymers, polymeric rubbers, polyolefin rubbers, elastomers, rubbers, thermoplastic elastomers, thermoset elastomers and elastomeric composites. In some embodiments, the flexibility of tether 34 includes movement in a lateral or side to side direction and prevents expanding and/or extension in an axial direction upon tensioning and attachment with a targeted portion of the anatomy. In some embodiments, all or only a portion of tether 34 may have a semi-rigid, rigid or elastic configuration, and/or have elastic properties, similar to the material examples described herein, such that tether 34 provides a selective amount of expansion and/or extension in an axial direction. In some embodiments, tether 34 may be compressible in an axial direction. Tether 34 can include a plurality of separately attachable or connectable portions or sections, such as bands or loops, or may be monolithically formed as a single continuous element.

In some embodiments, tether 34 may include a pliable lead such that tether 34 can be passed and/or guided through cavities of spinal tissue to resist and/or prevent non-desirable and/or harmful engagement with selected and/or sensitive anatomy of the spinal tissue. In some embodiments, the pliable lead is soft and flexible and configured to pass through a sub-laminar cavity of vertebrae without adhering to dura matter of a spinal cord and/or surfaces of a lamina of a vertebral level. In some embodiments, all or only a portion of the pliable lead is fabricated from a pliable, low-friction material, such as, for example, silicone, polyurethane, silicone-polyurethane copolymers, polymeric rubbers, polyolefin rubbers, elastomers, rubbers, thermoplastic elastomers, thermoset elastomers and elastomeric composites,

Tether 34 can have a uniform thickness/diameter. In some embodiments, tether 34 may have various surface configurations, such as, for example, smooth and/or surface configurations to enhance fixation, such as, for example, rough, arcuate, undulating, porous, semi-porous, dimpled, polished and/or textured. In some embodiments, the thickness defined by tether 34 may be uniformly increasing or decreasing, or have alternate diameter dimensions along its length. In some embodiments, tether 34 may have various cross section configurations, such as, for example, oval, oblong, triangular, rectangular, square, polygonal, irregular, uniform, non-uniform, variable and/or tapered. In some embodiments, the surface of tether 34 may include engaging structures, such as, for example, barbs, raised elements and/or spikes to facilitate engagement with tissue of the targeted anatomy.

In some embodiments, tether 34 may have various lengths. In some embodiments, tether 34 may be braided, such as a rope, or include a plurality of elongated elements to provide a predetermined force resistance. In some embodiments, tether 34 may be made from autograft and/or allograft, and be configured for resorbable or degradable applications. In one embodiment, tether 34 is a cadaver tendon. In one embodiment, tether 34 is a tendon that may be harvested, for example, from a patient or donor. In some embodiments, a tendon harvested from a patient may be affixed in remote locations with the patient's body.

In assembly, operation and use, spinal implant system 10, similar to the systems and methods described herein, is employed with a surgical procedure, such as, for example, a correction treatment of an affected portion of a spine, for example, a correction treatment to treat adolescent idiopathic scoliosis and/or Scheuermann's kyphosis of a spine. In some embodiments, one or all of the components of spinal implant system 10 can be delivered or implanted as a pre-assembled device or can be assembled in situ. Spinal implant system 10 may be completely or partially revised, removed or replaced.

In use, to treat a selected section of vertebrae V, including vertebrae V1, V2, as shown in FIG, 9, a medical practitioner obtains access to a surgical site including vertebrae V in any appropriate manner, such as through incision and retraction of tissues. In some embodiments, spinal implant system 10 can be used in any existing surgical method or technique including open surgery, mini-open surgery, minimally invasive surgery and percutaneous surgical implantation, whereby vertebrae V is accessed through a mini-incision, or a sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of spinal implant system 10. A preparation instrument (not shown) can be employed to prepare tissue surfaces of vertebrae V, as well as for aspiration and irrigation of a surgical region.

Tether 34 is delivered along the surgical pathway to a surgical site adjacent vertebrae V. End 90 of tether 34 is guided through opening 52 and cavity 36 for attachment with connector 12, as described herein. Tether 34 is disposed with vertebrae V, as described herein, to capture selected spinal tissue of vertebrae V, for example, tether 34 is wrapped about tissue of vertebra V1 and/or vertebra V2.

Base 18 is disposed in an initial non-locked orientation (FIG. 6) such that tether 34 is movable within cavity 36. End 92 is inserted into opening 52 and cavity 36 to form loop 94 about tissue of vertebra V1 and/or vertebra V2. In some embodiments, loop 94 is disposed about a transverse process of vertebra V2 by passing end 92 continuously about the transverse process. Loop 94 is fixed and/or attached with the transverse process and/or lamina. Tether 34 is threaded through opening 52 and cavity 36.

A surgical instrument, such as, for example, a tensioner T is disposed adjacent connector 12. Tensioner T is aligned and engaged with mating capture elements of connector 12 for releasable fixation and/or provisional fixation of tensioner T with connector 12. Tensioner T is actuated to tension tether 34 about vertebra V2. In some embodiments, the tension and/or tensile force applied to tether 34 and/or corrective forces applied to vertebrae V can be increased and/or decreased by tensioner T.

Tether 34 is slidably movable within cavity 36. Spinal rod 30 is delivered and inserted into bay 42, as shown in FIG, 7. Spinal rod 30 is delivered and inserted into bay 42 such that spinal rod 30 is engageable with surface 82 and/or surface 40 to splay surfaces 82, 40 to expand and/or open wall 80, in a direction shown by arrows B in FIG. 8. This configuration causes teeth 38, 54 to engage tether 34 and provisionally fix tether 34 with body 14.

Set screw 24 is engaged with support 16 via opening 22 in alignment with spinal rod 30. Set screw 24 is advanced, in the direction shown by arrow C in FIG. 8, for engaging spinal rod 30 such that spinal rod 30 translates within bay 42 to engage surface 82 with a force, in the direction shown by arrow D in FIG. 8. Spinal rod 30 engages surface 82 to rotate base 18 about hinge 48, in a direction shown by arrow E in FIG. 8. As set screw 24 is advanced and base 18 rotates, teeth 38, 54 engage tether 34 for fixation with surfaces 32, 50 via compressive forces and/or friction forces to dispose one or more components of spinal implant system 10 in a locked orientation. In some embodiments, set screw 24 is advanced and base 18 is selectively rotated such that teeth 38, 54 engage tether 34 for provisional fixation with surfaces 32, 50 to allow adjustment of one or more components of spinal implant system 10. In particular, prior to fixation of the components of spinal implant system 10, tether 34 may be further tightened.

Fixation of tether 34 with body 14 via rotation of base 18 and engagement of teeth 38, 54 with tether 34 fixes spinal rod 30, connector 12 and tether 34 in a selectively locked orientation in connection with the surgical treatment. Engagement of set screw 24 with spinal rod 30 fixes spinal rod 30 with body 14 such that spinal rod 30 and tether 34 are locked and/or disposed in a fixed orientation with body 14 and relative to connector 12 adjacent vertebra V2. This configuration tensions tether 34 about vertebra V2 and tensions the spinal construct for attachment with vertebrae V and/or to apply corrective treatment to vertebrae V.

In some embodiments, spinal implant system 10 includes a second spinal rod (not shown) delivered along the surgical pathway to the surgical site adjacent a contra-lateral side of vertebrae V. The second spinal rod is connected with the contra-lateral side of vertebrae V via one or more tethers 34, similar to spinal rod 30 described herein. Spinal rod 30 and the second spinal rod are fixed with vertebrae V in a side by side orientation and/or a bi-lateral arrangement to stabilize vertebrae V and affect growth for a correction treatment to treat spine pathologies, as described herein. In some embodiments, one or all of the components of spinal implant system 10 can be delivered or implanted as a pre-assembled device or can be assembled in situ, in a selected order of assembly or the order of assembly of the particular components of system 10 can be varied according to practitioner preference, patient anatomy or surgical procedure parameters.

Upon completion of the procedure, the surgical instruments, assemblies and non-implanted components of spinal implant system 10 are removed from the surgical site and the incision is dosed. One or more of the components of spinal implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation with vertebrae. In some embodiments, the agent may include one or a plurality of therapeutic agents and/or pharmacological agents for release, including sustained release, to treat, for example, pain, inflammation and degeneration.

In some embodiments, the components of spinal implant system 10 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of spinal implant system 10 may be used to prevent or minimize curve progression in individuals of various ages.

It will be understood that various modifications may be made to the embodiments disclosed herein. Therefore, the above description should not be construed as limiting, but merely as exemplification of the various embodiments. Those skilled in the art will envision other modifications within the scope and spirit of the dams appended hereto.

1. A spinal implant comprising:
a body including a support and a base that define a cavity configured for disposal of a member and including a wall that defines a bay configured for disposal of a longitudinal element, the support defining a threaded opening that is in communication with the bay,
the longitudinal element being engageable with the wall to fix the longitudinal element with the body and move the base relative to the support to fix the member with the body in the cavity.

2. A spinal implant as recited in claim 1, wherein the base is rotatable relative to the support.

3. A spinal implant as recited in claim 1, wherein the base is hinged to the support and rotates relative thereto.

4. A spinal implant as recited in claim 1, wherein the base is rotatable relative to the support between a non-locking orientation such that the member is movable relative to the body, and a locked orientation such that the member is disposed between the base and the support to fix the member with the body.

5. A spinal implant as recited in claim 1, wherein the cavity is separate from the bay.

6. A spinal implant as recited in claim 1, wherein the cavity is separate and oriented transverse relative to the bay.

7. A spinal implant as recited in claim 1, wherein at least one of the support and the base include a fixation surface engageable with the member to fix the member with the body.

8. A spinal implant as recited in claim 1, wherein the support and the base each include teeth engageable with the member to fix the member with the body.

9. A spinal implant as recited in claim 1, wherein the member includes a pliable lead.

10. A spinal implant as recited in claim 1, wherein the bay is expandable.

11. A spinal implant as recited in claim 1, further comprising a coupling member disposable within the threaded opening and engageable with the longitudinal element to engage the longitudinal element with the wall.

12. A spinal implant as recited in claim 1, wherein the longitudinal element is engageable with the wall in a snap-on configuration to dispose the longitudinal element in the bay.

13. A spinal implant as recited in claim 1, wherein the support includes a finger engageable with the longitudinal element to provisionally dispose the longitudinal element in the bay.

14. A spinal implant comprising:
a body including a support and a base that define a cavity configured for disposal of a member and include a wall that defines a bay configured for disposal of a longitudinal element, the support defining a threaded opening that is in communication with the bay,
the longitudinal element being engageable with the wall such that the base is rotatable relative to the support between a non-locking orientation such that the member is translatable relative to the body and a locked orientation such that the member is disposed between the base and the support to fix the member with the body.

15. A spinal implant system comprising:
a connector including a support and a base that define a cavity and include a wall that defines a bay, the support defining a threaded opening that is in communication with the bay;
a tether disposable in the cavity; and
a spinal rod disposable in the bay,
the spinal rod being engageable with the wall to fix the spinal rod with the connector and move the base relative to the support to fix the tether with the connector in the cavity.

16. A spinal implant system as recited in claim 15, wherein the base is rotatable relative to the support.

17. A spinal implant system as recited in claim 15, wherein the base is rotatable relative to the support between a non-locking orientation such that the tether is translatable relative to the connector, and a locked orientation such that the tether is disposed between the base and the support to fix the tether with the connector.

18. A spinal implant system as recited in claim 15, wherein the support and the base each include teeth engageable with the tether to fix the tether with the connector.

19. A spinal implant system as recited in claim 15, wherein the tether includes a pliable lead.

20. A spinal implant system as recited in claim 19, wherein the bay is expandable.
